(19) United States
(12) Patent Application Publication
(10) Pub. No.: US 2013/0079754 A1
(43) Pub. Date: Mar. 28, 2013

(54) CONNECTION SYSTEM

(71) Applicant: Garrett Austin Sheffer, Totowa, NJ (US)

(72) Inventor: Garrett Austin Sheffer, Totowa, NJ (US)

(73) Assignee: GENERAL ELECTRIC COMPANY, Schenectady, NY (US)

(21) Appl. No.: 13/245,371

(22) Filed: Sep. 26, 2011

(51) Int. Cl. A61M 25/16

(52) U.S. Cl. 604/535

(57) ABSTRACT

A connection system is disclosed herein in accordance with an embodiment. The connection system includes a male component and a female component. At least one of the male component and the female component may define a tapered surface. The connection system also includes a keyed interface disposed on either the male component or the female component. The keyed interface is configured to prevent the male component and the female component from unintentionally being coupled with a non-conforming connection system component.

### FIELD OF THE INVENTION

This disclosure relates to a connection system. More precisely, this disclosure relates a connection system with a keyed interface.

### BACKGROUND OF THE INVENTION

Tapered connection systems such as the Luer-Lok and the Luer-Slip connections may be implemented to establish fluid couplings. Tapered connection systems generally comprise male and female components with a complementary geometry that are adapted to fit together and form a leak-free connection. Tapered connection systems are commonly implemented to connect hypodermic syringe tips with a syringe barrel. Tapered connection systems are also commonly implemented for a variety of other applications such as for intravenous tubing connections, blood pressure cuff/tubing connections, anesthesia machine couplings, etc.

One problem with conventional tapered connection systems is that they do not provide adequate error protection. As an example, a male connection component adapted to transfer oxygen could inadvertently be connected with a female connection component adapted to receive intravenous fluid because of the common geometries.

### BRIEF DESCRIPTION OF THE INVENTION

The above-mentioned shortcomings, disadvantages and problems are addressed herein which will be understood by reading and understanding the following specification.

In an embodiment, a connection system includes a male component and a female component adapted to receive the male component. At least one of the male component and the female component may define a tapered surface. The connection system also includes a keyed interface disposed on either the male component or the female component. The keyed interface is configured to prevent the male component and the female component from unintentionally being coupled with a non-conforming connection system component.

In another embodiment, a tapered connection system includes a male component defining an external tapered surface, and a female component defining an internal tapered surface. The internal tapered surface is complimentary with and adapted to receive the external tapered surface. One of the male component and the female component comprises a keyed protrusion. The keyed protrusion is configured to prevent unintentional coupling with a non-conforming connection system component. The other of the male component and the female component comprises a keyed recession complementary with and adapted to receive the keyed protrusion.

Various other features, objects, and advantages of the invention will be made apparent to those skilled in the art from the accompanying drawings and detailed description thereof.

### BRIEF DESCRIPTION OF THE DRAWINGS

FIG. 1 is a schematic representation of a connection system in accordance with an embodiment;

FIG. 2 is a detailed view of the connection system components of FIG. 1 in accordance with an embodiment;

FIG. 3 is a detailed view of the connection system components of FIG. 1 in accordance with another embodiment;

FIG. 4 is a detailed view of the connection system components of FIG. 1 in accordance with another embodiment; and

FIG. 5 is a detailed view of the connection system components of FIG. 1 in accordance with another embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

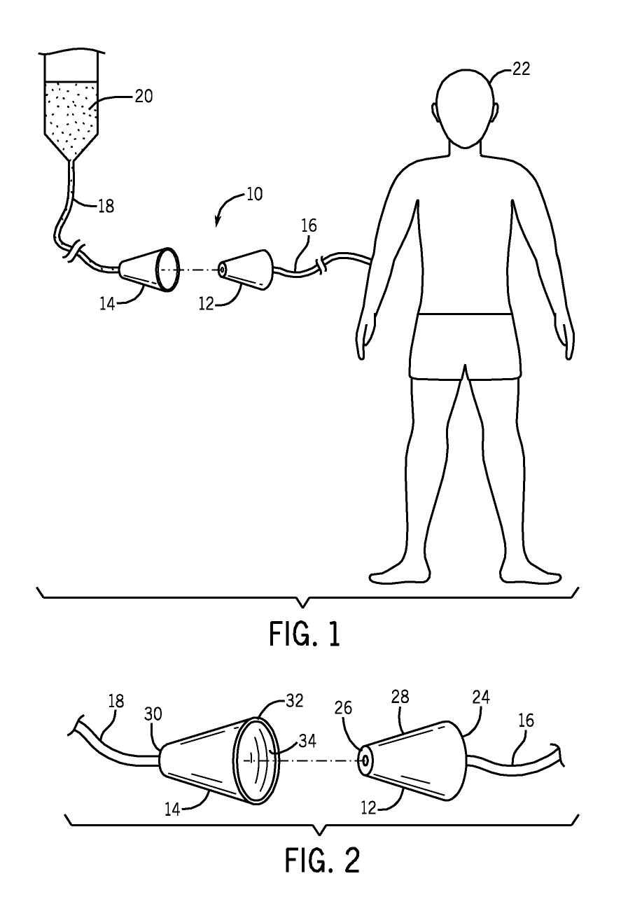

Referring to FIG. 1, a tapered connection system 10 is schematically depicted in accordance with an embodiment. The tapered connection system 10 comprises a male connector or component 12 and a female connector or component 14. The tapered connection system 10 may be implemented to form a fluid coupling. For purposes of this disclosure the term fluid should be defined to include any substance that continually deforms under an applied shear stress and may therefore include both liquids and gases. According to one embodiment the tapered connection system 10 comprises a Luer tapered connection such as a Luer-Lok or Luer-Slip connection; however a variety of alternate tapered connection systems may be envisioned.

The tapered connection system 10 will be described in accordance with an exemplary embodiment as being adapted to couple a first intravenous (IV) tube 16 with a second IV tube 18. The tubes 16, 18 may be implemented to deliver an IV fluid 20 to a patient 22. It should be appreciated that the tapered connection system 10 may be implemented in a variety of different applications and/or with a variety of different devices.

Referring to FIG. 2, the male connector 12 may generally define a truncated conical geometry. The male connector 12 may comprise a proximal end 24 secured to the IV tube 16. The male connector 12 may also comprise a distal end 26, and a tapered external surface 28 defined on the exterior of the male connector 12 between the proximal end 24 and the distal end 26. The tapered external surface 28 preferably tapers in a generally continuous manner such that the outer diameter becomes smaller toward the distal end 26.

The female connector 14 may comprise a proximal end 30 secured to the IV tube 18. The female connector 14 may also comprise a distal end 32, and a tapered internal surface 34 defined on the interior of the female connector 14 between the proximal end 30 and the distal end 32. The tapered internal surface 34 preferably tapers in a generally continuous manner such that the inner diameter becomes larger toward the distal end 32. The tapered surfaces 34, 28 have been described in accordance with an embodiment. It should, however, be appreciated that alternatively only one of the male connector 12 and the female connector 14 may comprise a tapered surface. As an example, the female connector 14 may comprise the tapered internal surface 34 adapted to receive a cylindrical or square male connector defining an external surface (not shown) that is not tapered.

The geometries of the tapered external surface 28 and the tapered internal surface 34 are preferably complementary. As an example, the tapered internal surface 34 of the female connector 14 may be configured to receive the tapered external surface 28 of the male connector 12 such that upon insertion the surfaces 34, 28 engage to establish a sealed connection. Alternatively, the sealed connection can be established based on a point or line contact, or any combination thereof, rather than or in addition to the previously described surface engagement. The sealed connection may be maintained in a known manner such as, for example, based on friction and/or material deformation.

Material properties such as durometer and elasticity may be selectable to help form the sealed connection.

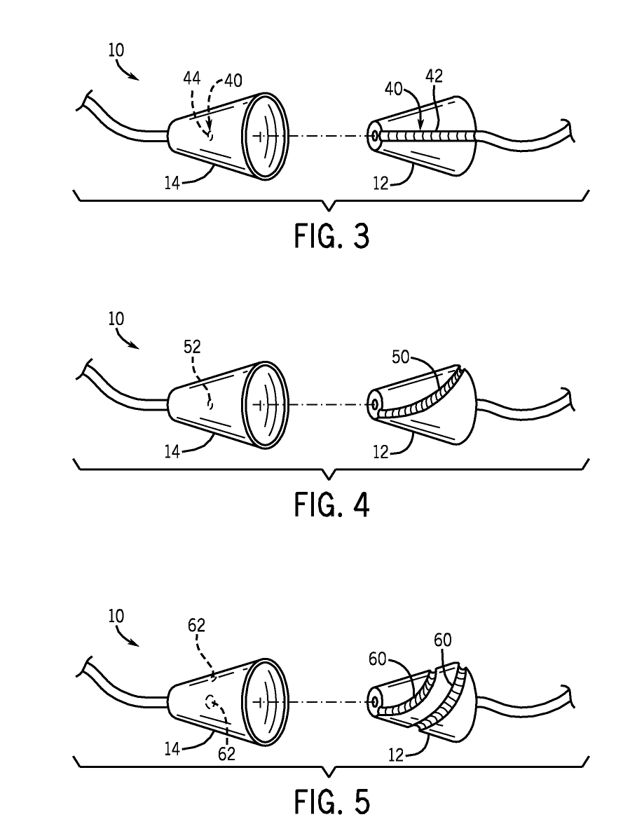

Referring to FIGS. 3-5, the tapered connection system 10 comprises a keyed interface 40 adapted to reduce the likelihood of unintentionally coupling inappropriate connection system components. The keyed interface 40 may include a keyed recession 42 and a keyed protrusion 44. For illustrative purposes, the male connector 12 will be described as comprising the keyed recession 42 and the female connector 14 will be described as comprising the keyed protrusion 44. Alternatively, the male connector 12 may comprise the keyed protrusion 44 and the female connector 14 may comprise the keyed recession 42.

Referring to FIG. 3, the depicted keyed recession 42 comprises an elongated generally linear groove 42. The elongated groove 42 comprises a concave arcuate or semi-circular cross-section. The depicted keyed protrusion 44 comprises a tab or pin 44. The tab 44 comprises convex arcuate or semi-circular cross-section. It should be appreciated that other cross-section geometries for the groove 42 and tab 44 may be envisioned such as, for example, square; rectangular; v-shaped; u-shaped; etc. The tab 44 may comprise a generally discrete protrusion as depicted or may alternatively comprise an elongated ridge (not shown).

The geometries of the keyed recession 42 and the keyed protrusion 44 are preferably complementary. According to one embodiment, the keyed recession 42 may be configured to receive a keyed protrusion 44 with a common cross sectional geometry and with minimal clearance. The keyed protrusion 44 is adapted to interrupt engagement with tapered connection system components that do not include a complementary keyed recession 42 in order to limit the unintentional coupling of non-conforming components. The term non-conforming component may be defined as any tapered connection system component without an appropriate complimentary keyed interface feature.

According to a first alternative embodiment, the generally linear groove 42 may define an L-shaped or J-shaped geometry (not shown). The L-shaped or J-shaped geometry may be configured to receive the tab 44 during an initial linear insertion of the male connector 12 into the female connector 14, and to then allow for rotation of the male connector 12 relative to the female connector 14 to form a more secure connection.

According to a second alternative embodiment, the generally linear groove 42 may comprise a unique axial length adapted to just accommodate a correspondingly unique axially positioned tab 44. The axial length of the linear groove 24 and the corresponding axial position of the tab 44 may be implemented to help limit the unintentional coupling of non-conforming components. As an example, if a non-conforming component with a linear groove having a shorter axial length were unintentionally implemented, the axial position of the tab 44 would limit component insertion and thereby prevent the coupling.

FIG. 4 is an alternative embodiment of a tapered connection system 10. It should be noted that throughout the exemplarily embodiments disclosed herein, like reference numerals are used to described like structures between the embodiments.

The depicted keyed recession 50 comprises an arcuate or helical groove 50 but is otherwise similar to the keyed recession 42. The keyed recession 50 is preferably complementary with and adapted to receive the keyed protrusion 52. The helical geometry of the groove 50 causes the male connector 12 to twist relative to the female connector 14 during insertion. The relative twisting may tighten the tapered connection so that it is less likely to leak.

FIG. 5 is an alternative embodiment of a tapered connection system 10. The depicted keyed recession 60 comprises multiple arcuate or helical grooves 60 but is otherwise similar to the keyed recession 50. Correspondingly, the keyed protrusion 62 comprises multiple tabs 62. The grooves 60 are complimentary with and adapted to accommodate the tabs 62. The multiple grooves 60 may differ in size or shape from each other as long as the corresponding tabs 62 remain complementary.

As a non-limiting example, one of the multiple grooves 60 may be relatively narrower with a concave semi-circular cross-section, while the other of the grooves 60 may be relatively wider with a concave u-shaped cross-section. Further in accordance with this non-limiting example, one of the multiple tabs 62 would be relatively smaller with a convex semi-circular cross-section, while the other of the tabs 62 would be relatively larger with a convex u-shaped cross-section. The implementation of multiple grooves 60 and multiple complementary tabs 62 with variable geometries reduces the likelihood that non-conforming components will unintentionally be coupled.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. A connection system comprising:
a male component;
a female component adapted to receive the male component, at least one of the male component and the female component comprising a tapered surface; and
a keyed interface disposed on one of the male component and the female component, said keyed interface being configured to prevent the male component and the female component from unintentionally being coupled with a non-conforming connection system component.

2. The connection system of claim 1, wherein the keyed interface comprises a keyed protrusion and a keyed recession.

3. The connection system of claim 2, wherein the keyed recession comprises a generally linear groove.

4. The connection system of claim 2, wherein the keyed recession comprises a generally helical groove.

5. The connection system of claim 2, wherein the keyed recession comprises a plurality of grooves.

6. The connection system of claim 2, wherein the keyed protrusion comprises a discrete tab.

7. The connection system of claim 2, wherein the keyed protrusion comprises an elongated ridge.

8. The connection system of claim 2, wherein the keyed protrusion comprises a cross-section defining a convex geometry, and wherein the keyed recession comprises a cross-section defining a complementary concave geometry.

9. The connection system of claim 1, wherein the connection system comprises a Luer-Lok or Luer-Slip connection.

10. A tapered connection system comprising:
a male component defining an external tapered surface; and
a female component defining an internal tapered surface, said internal tapered surface complimentary with and adapted to receive the external tapered surface;
wherein one of said male component and said female component comprises a keyed protrusion, said keyed protrusion configured to prevent unintentional coupling with a non-conforming connection system component;
wherein the other of said male component and said female component comprises a keyed recession complementary with and adapted to receive the keyed protrusion.

11. The tapered connection system of claim 10, wherein the keyed recession comprises a generally linear groove.

12. The tapered connection system of claim 10, wherein the keyed recession comprises a generally helical groove.

13. The tapered connection system of claim 10, wherein the keyed recession comprises a plurality of grooves.

14. The tapered connection system of claim 10, wherein the tapered connection system comprises a Luer-Lok or Luer-Slip connection.
